(19) United States
(12) Patent Application Publication
(10) Pub. No.: US 2022/0096862 A1
(43) Pub. Date: Mar. 31, 2022

(54) METHOD FOR IRRADIATING CELLS WITH LIGHT, METHOD FOR CONTROLLING MEDICAL DEVICE, AND MEDICAL DEVICE

(71) Applicant: OLYMPUS CORPORATION, Tokyo (JP)

(72) Inventors: Masahiro YOSHINO, Tokyo (JP); Nobuhiko ONDA, Kanagawa (JP); Susumu YAMASHITA, Tokyo (JP); Miho KOJIMA, Tokyo (JP)

(73) Assignee: OLYMPUS CORPORATION, Tokyo (JP)

(21) Appl. No.: 17/550,725

(22) Filed: Dec. 14, 2021

Related U.S. Application Data
(63) Continuation of application No. PCT/JP2019/035255, filed on Sep. 6, 2019.

(51) Int. Cl. A61N 5/06; A61K 41/00

(52) U.S. Cl. CPC A61N 5/062; A61N 2005/0659; A61K 41/008

(57) ABSTRACT

A method for irradiating cells with light includes administering a photosensitizing agent that uses a phthalocyanine dye, which is a fluorescent dye, and irradiating cells with therapeutic light with a light intensity of more than 0 mW/cm² and 50 mW/cm² or less up to at least 1 J/cm².

### CROSS REFERENCE TO RELATED APPLICATION

This application is a continuation application of PCT/JP2019/035255 filed on Sep. 6, 2019, the entire contents of which are incorporated herein by this reference.

### BACKGROUND OF THE INVENTION

### 1. Field of the Invention

The present invention relates to a method for irradiating cells with light that includes a step of administering a photosensitizing agent to cells and a step of irradiating the cells with predetermined light, to a method for controlling a medical device, and to a medical device.

### 2. Description of the Related Art

There is a well-known technique, known as Photodynamic Therapy (PDT), where an agent containing an oncotropic photosensitive substance is administered into a body, and the oncotropic photosensitive substance is irradiated with light to kill cancer cells.

The oncotropic photosensitive substance administered into the body has the property of collecting in cancer cells rather than in normal cells and has the property of being activated and producing active oxygen with irradiation with a laser beam. In PDT, by making use of such properties of the oncotropic photosensitive substance, cancer cells are killed by chemical reactions of the oncotropic photosensitive substance collecting in the cancer cells by controlling the location of the irradiation with light.

Japanese Patent Application Laid-Open Publication No. 2017-71654 discloses a technique, known as Photo immunotherapy (PIT), where a photosensitizing agent is administered into the body, and irradiated with near infrared light to kill (break up) cancer cells. The photosensitizing agent is a phthalocyanine fluorescent dye (IRDye 700) conjugated to a targeting molecule (antibody) that binds a protein on cell.

The photosensitizing agent administered into the body has the property of attaching specifically to protein of cancer cells. In PIT, by making use of such properties of the photosensitizing agent, the photosensitizing agent is activated by irradiation with near infrared light having wavelength of 660 to 710 nm up to at least 1 J/cm².

As a result, the photosensitizing agent absorbs light to generate energy for damaging cells. A detailed principle, a method, and various conditions of PIT are disclosed in Japanese Patent Application Laid-Open Publication No. 2017-71654, for example.

### SUMMARY OF THE INVENTION

A method for irradiating cells with light according to one aspect of the present invention includes, administering a photosensitizing agent including a phthalocyanine fluorescent dye to cells: and irradiating the cells with light with a light intensity of more than 0 mW/cm² and 50 mW/cm² or less up to at least 1 J/cm².

A method for controlling a medical device according to one aspect of the present invention is a method for controlling a medical device capable of irradiating cells with therapeutic light, the method including irradiating cells to which a photosensitizing agent including a phthalocyanine fluorescent dye is administered with predetermined light with a light intensity of more than 0 mW/cm² and 50 mW/cm² or less up to at least 1 J/cm².

A medical device according to one aspect of the present invention includes: a therapeutic light source configured to irradiate cells to which a photosensitizing agent including a phthalocyanine fluorescent dye is administered with predetermined light with a light intensity of more than 0 mW/cm² and 50 mW/cm² or less; and a therapeutic light irradiation device configured to control the therapeutic light source to perform irradiation with the predetermined light up to at least 1 J/cm².

### BRIEF DESCRIPTION OF THE DRAWINGS

FIG. 1 is a view showing a light irradiation system used in a method for irradiating cells with light of a present embodiment;

FIG. 2 is a view showing a schematic configuration of a photosensitizing agent administered to the cells shown in FIG. 1;

FIG. 3 is a flowchart showing a therapeutic light irradiation method that uses the light irradiation system shown in FIG. 1;

FIG. 4 is a chart showing a relationship between a percentage of attenuation of fluorescence and cellular cytotoxicity when cells are irradiated with therapeutic light with a light intensity of 50 mW/cm² or less and therapeutic light with a light intensity of 100 mW/cm² or more;

FIG. 5 is a chart showing a relationship between a light intensity and cellular cytotoxicity what the cells are irradiated with therapeutic light with a light intensity of 25 mW/cm², therapeutic light with a light intensity of 50 mW/cm², therapeutic light with a light intensity of 100 mW/cm², and therapeutic light with a light intensity of 300 mW/cm²; and

FIG. 6 is a flowchart showing a modification where a fluorescence intensity measurement step shown in FIG. 3 is performed after the cells are irradiated with therapeutic light up to a predetermined amount of light.

### DETAILED DESCRIPTION OF THE PREFERRED EMBODIMENT

Hereinafter, an embodiment of the present invention will be described with reference to drawings.

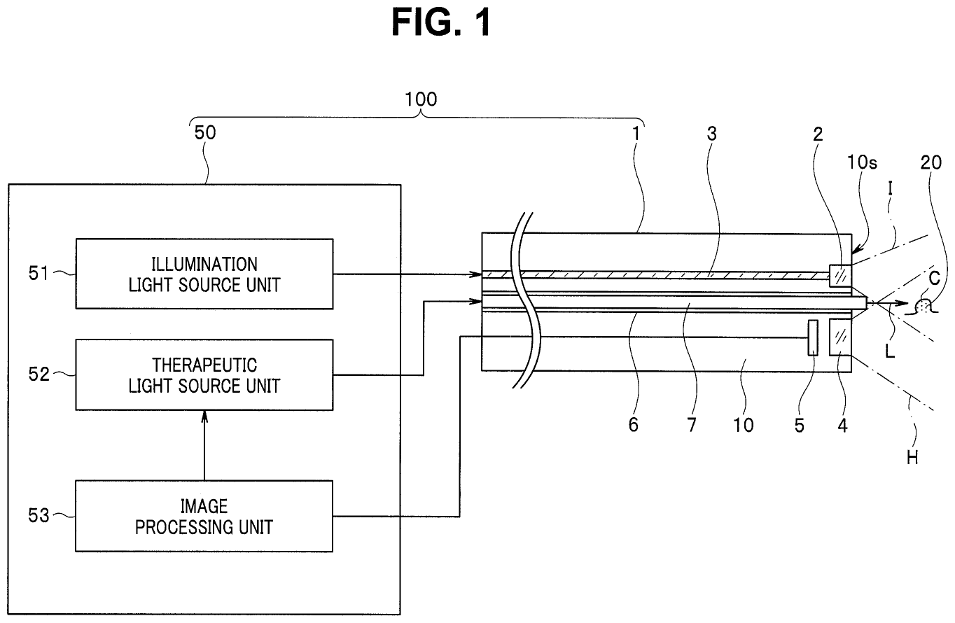

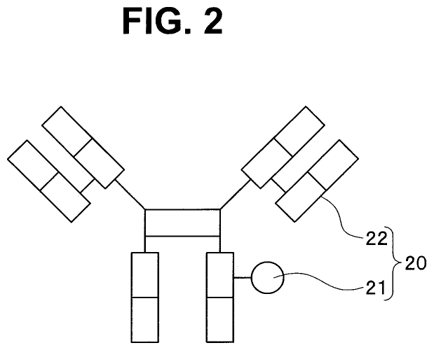

FIG. 1 is a view showing a light irradiation system used in a method for irradiating cells with light of the present embodiment. FIG. 2 is a view showing a schematic configuration of a photosensitizing agent to be administered to the cells shown in FIG. 1.

As shown in FIG. 1, a main part of a light irradiation system 100 used in the above-mentioned PIT is formed of an endoscope 1 and a processor 50.

The endoscope 1 includes an insertion portion 10 to be inserted into a subject. A distal end surface 10s of the insertion portion 10 is provided with an objective optical system 4 and an illumination optical system 2 such that the objective optical system 4 and the illumination optical system 2 face the distal end surface 10s. The objective optical system 4 observes an observation range H in the subject. The illumination optical system 2 supplies illumination light 1 into the subject.

An image pickup device 5 is provided in the insertion portion 10 at a position where the objective optical system 4 forms an image.

A light guide 3 is also provided in the insertion portion 10, and the light guide 3 supplies the illumination light I to the illumination optical system 2. A configuration of supplying the illumination light I into the subject may use light emitting elements, such as LEDs.

The insertion portion 10 is provided with a channel 6 that is opened on the distal end surface 10s, and a therapeutic light irradiation device 7 can be inserted into and removed from the channel 6.

The therapeutic light irradiation device 7 is configured to be inserted into the channel 6 from a proximal-end-side insertion port not shown in the drawing of the channel 6 to irradiate cancer cells in the subject (hereinafter simply referred to as “cells”) C to which a photosensitizing agent 20 is administered with therapeutic light L, which is predetermined light, in a state where the therapeutic light irradiation device 7 is caused to protrude into the subject from a distal end of the channel 6. Note that the therapeutic light is light to activate a photosensitizing agent for treatment.

An example of the therapeutic light L may be near infrared light. An example of the photosensitizing agent 20 may be Pan-IR700, which is obtained by causing, as shown in FIG. 2, a phthalocyanine fluorescent dye (IRDye 700) 21 to label with (attach to) one antibody molecule (panitumumab, monoclonal antibody to Human EGFR) 22. The photosensitizing agent 20 is not limited to Pan-IR700, but is only required to be a photosensitizing agent using a phthalocyanine fluorescent dye.

A configuration may also be adopted where the cells C are irradiated with the therapeutic light L by using the light guide 3 and the illumination optical system 2 without using the therapeutic light irradiation device 7.

The processor 50 includes an illumination light source unit 51, a therapeutic light source unit 52, and an image processing unit 53.

The illumination light source unit 51 is configured to supply the illumination light I to the light guide 3 to supply the illumination light I to the illumination optical system 2.

The therapeutic light source unit 52 is configured to supply the therapeutic light L to the therapeutic light irradiation device 7. The therapeutic light source unit 52 is electrically connected to the image processing unit 53, and supplies the therapeutic light L to the therapeutic light irradiation device 7 based on image determination which is described later and performed by the image processing unit 53.

The image processing unit 53, which is a control unit of the present embodiment, is electrically connected to the image pickup device 5. The image processing unit 53 measures, by using an image of the cells C picked up by the image pickup device 5, intensity data of fluorescence that is generated from the photosensitizing agent 20 with the irradiation of the cells C with the therapeutic light L. After the image processing unit 53 compares the intensity data with a predetermined value, the image processing unit 53 determines whether to cause the therapeutic light source unit 52 to continuously perform irradiation with the therapeutic light L.

The therapeutic light source unit 52 may be either incorporated in the processor 50 or externally provided to the processor 50.

In the case where the therapeutic light source unit 52 is externally provided to the processor 50, the intensity data of the fluorescence may be displayed on a monitor not shown in the drawing, and an operator may determine, based on the intensity data of the fluorescence displayed on the monitor, whether irradiation with the therapeutic light L is to be continuously performed.

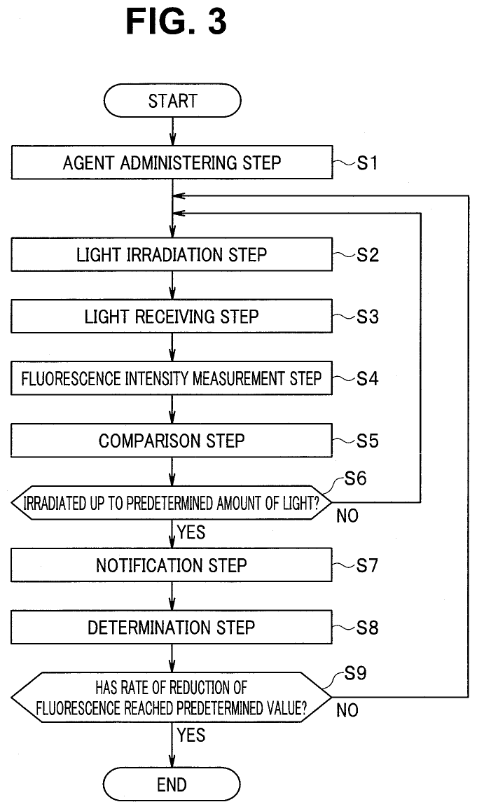

Next, a method for irradiating, by using the light irradiation system 100 shown in FIG. 1, the photosensitizing agent 20 administered to the cells C with the therapeutic light L at the time of performing PIT will be described with reference to FIG. 3.

FIG. 3 is a flowchart showing a therapeutic light irradiation method that uses the light irradiation system shown in FIG. 1.

In performing PIT, as shown in FIG. 3, first, an agent administering step is performed in step S1. In the agent administering step, the photosensitizing agent 20 shown in FIG. 2 is administered to the cells C.

More specifically, depending on the location of the ceils C in the subject, the photosensitizing agent 20 is administered within the observation range H of the objective optical system 4 of the endoscope 1, for example, via a systemic route, a topical route, an intravenous route, an intraperitoneal route, an oral route, an ocular route, a sublingual route, a rectal route, a transdermal route, an intranasal route, a vaginal route, an inhalation route or other routes A technique of administering the photosensitizing agent 20 is not limited to a technique that uses the endoscope 1.

Next, a light irradiation step is performed in step S2. In the light irradiation step, in a state where the illumination light I is supplied from the illumination optical system 2 to the cells C in the subject and within the observation range H of the objective optical system 4, the cells C are irradiated from the therapeutic light source unit 52 with the therapeutic light L with a light intensity (irradiation power density) of more than 0 mW/cm² and 50 mW/cm² or less by using the therapeutic light irradiation device 7 up to at least 1 J/cm². Such irradiation with the therapeutic light L may be controlled by the light source unit per se, or may be controlled by a doctor.

The reason for irradiating the cells C with the therapeutic light L up to at least 1 J/cm² is based on a condition of a minimum total amount of irradiation at which a therapeutic effect is exhibited and which is disclosed in Japanese Patent Application Laid-Open Publication No. 2017-71654. The reason for setting the light intensity to more than 0 mW/cm² and 50 mW/cm² or less will be described later.

Hereinafter, the description will be made with respect to a technique for estimating, in step S3 and following steps, a percentage of damage occurrence, being a percentage of the cells C being killed or a percentage of cells being damaged (hereinafter, referred to as “cellular cytotoxicity”).

The reason is as follows. When the photosensitizing agent 20 is irradiated with near infrared light, which is the therapeutic light L, at the time of absorbing light, the photosensitizing agent 20 emits fluorescence as well as energy for damaging the cells. The emitted fluorescence attenuates with irradiation by the therapeutic light L. Therefore, it is possible to estimate cellular cytotoxicity by monitoring a rate of reduction of fluorescence of the photosensitizing agent 20 during PIT or after PIT

Thereafter, after the cells C are irradiated with the therapeutic light L, a light receiving step is performed in step S3. In the light receiving step, the image pickup device 5 receives fluorescence from the cells C by using the objective optical system 4, the fluorescence being generated from the photosensitizing agent 20 with the therapeutic light L.

Next, a fluorescence intensity measurement step is performed in step S4. In the fluorescence intensity measurement step, measurement is made of intensity data of the fluorescence that is received by the image processing unit 53 after irradiation with the therapeutic light L is started.

In the present embodiment, in the fluorescence intensity measurement step, the intensity data of the fluorescence are acquired during treatment, which is irradiation of the cells C with the therapeutic light L.

Thereafter, a comparison step is performed in step S5. In the comparison step, the image processing unit 53 compares the intensity data of the fluorescence with the predetermined value.

More specifically, the comparison step is performed where the image processing unit 53 makes a comparison, based on acquired intensity data of the fluorescence, to determine whether a percentage of attenuation of fluorescence (a rate of reduction of fluorescence) exceeds the predetermined value of, for example, approximately 70%.

Next, in step S6, the image processing unit 53 determines whether the cells C are irradiated from the therapeutic light source unit 52 with the therapeutic light L by a predetermined amount of light, more specifically, up to at least 1 J/cm².

When the cells C are not irradiated up to at least 1 J/cm², step S2 to step S6 are repeated.

In contrast, when the cells C are irradiated with the therapeutic light L up to at least 1 J/cm², the process shifts to step S7 where a notification step is performed. In the notification step, the operator is notified that the cells C are irradiated with the therapeutic light L up to 1 J/cm². Examples of a specific notification method may be a known sound, light, display and the like.

In following step S8, a determination step is performed. In the determination step, after irradiation with the therapeutic light L, when the rate of reduction of fluorescence acquired from the intensity data is less than approximately 70%, the image processing unit 53 determines that the cells C are to be continuously irradiated with the therapeutic light L until the rate of reduction becomes approximately 70%, and gives an instruction to the therapeutic light source unit 52. In contrast, when the rate of reduction of fluorescence reaches approximately 70%, the image processing unit 53 determines that further irradiation with the therapeutic light L is unnecessary.

In following step S9, when the rate of reduction of fluorescence does not reach approximately 70%, it is determined that the therapeutic effect is low, and the process returns to step S2 and step S2 to step S9 are repeated.

In contrast, when the rate of reduction of fluorescence reaches approximately 70%, it is determined that the therapeutic effect is achieved with respect to the cells C, that is, the cells C are killed. Therefore, it is assumed that treatment is finished, and irradiation with the therapeutic light L is finished.

As described above, the percentage of the cells C being killed can be inferred from the acquired rate of reduction of fluorescence. In other words, it is possible to monitor the percentage of the cells C being killed by monitoring the rate of reduction of fluorescence.

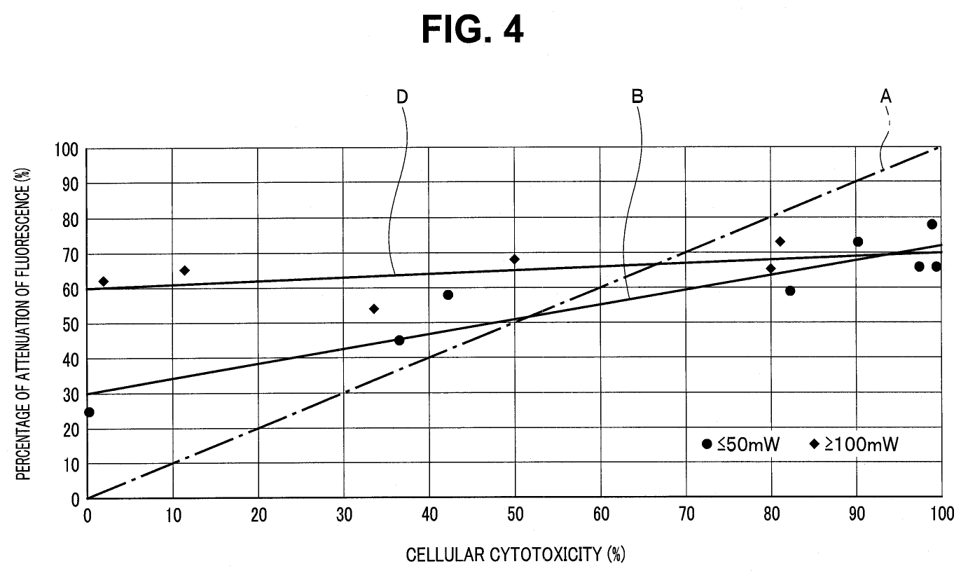

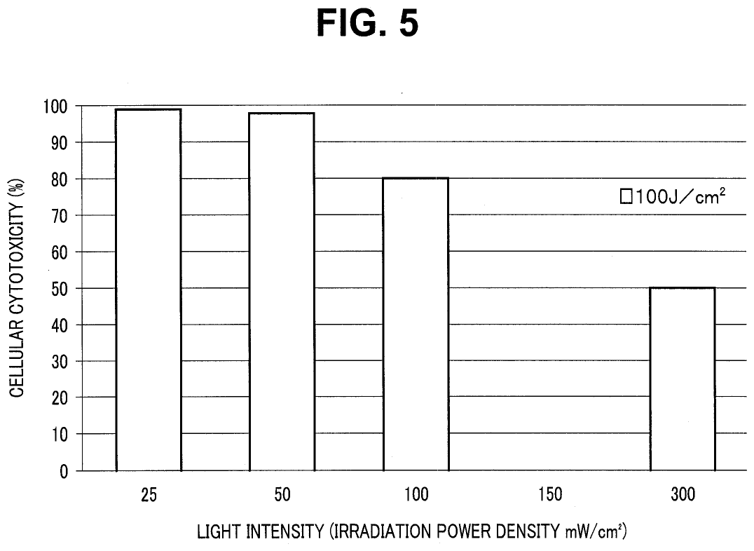

Next, the reason that the light intensity with respect to the cells C is set to more than 0 mW/cm² and 50 mW/cm² or less in the light irradiation step in step S2 shown in FIG. 3 and the basis for setting the rate of reduction of fluorescence used in the comparison to approximately 70% in the comparison step in step S5 and in the determination step in step S8 and step S9 shown in FIG. 3 are described by using FIG. 4 and FIG. 5.

FIG. 4 is a chart showing a relationship between the percentage of attenuation of fluorescence and cellular cytotoxicity when the cells are irradiated with therapeutic light with a light intensity of 50 mW/cm² or less and therapeutic light with a light intensity of 100 mW/cm² or more. FIG. 5 is a chart showing a relationship between a light intensity and cellular cytotoxicity when the cells are irradiated with therapeutic light with a light intensity of 25 mW/cm², therapeutic light with a light intensity of 50 mW/cm², therapeutic light with a light intensity of 100 mW/cm², and therapeutic light with a light intensity of 300 mW/cm².

First, the chart of experimental data shown in FIG. 4 shows a comparison in a case where Pan-IR700, which is the photosensitizing agent 20, is administered to A431 tumor bearing mice, and the A431 tumor bearing mice are respectively irradiated with the therapeutic light L with a light intensity of 50 mW/cm² or less and therapeutic light with a light intensity of 100 mW/cm² or more.

The total amount of irradiation is the same in both the case where irradiation is performed with the therapeutic light L with the light intensity of 50 mW/cm² or less and the case where irradiation is performed with the therapeutic light L with the light intensity of 100 mW/cm² or more. In other words, in the case where irradiation is performed with the therapeutic light L with the light intensity of 50 mW/cm² or less, a time period during which the cells C are irradiated is longer than a time period during which the cells C are irradiated with the therapeutic light L with the light intensity of 100 mW/cm² or more.

After the cells C are irradiated with the therapeutic light L, if the rate of reduction of fluorescence (the percentage of attenuation of fluorescence) and cellular cytotoxicity have a proportional relation, as shown by a dashed-and-dotted line A in FIG. 4, when the rate of reduction of fluorescence is 100%, cellular cytotoxicity is 100%. Therefore, it should be determined that all cells C disappear if fluorescence is no longer detected.

How ever, as the result of experiments performed by the applicant, the following is found. In actual experiments, as the result of observation that is performed after irradiation, by using the endoscope 1, for example, as the rate of reduction of fluorescence approaches 70% (or little more than 70%), cellular cytotoxicity approaches 100%. A solid line B shows a case where irradiation is performed with the therapeutic light L with the light intensity of 50 mW/cm² or less. A solid line D shows a case where irradiation is performed with the therapeutic light L with the light intensity of 100 mW/cm² or more.

This is the basis for setting the rate of reduction of fluorescence used in the comparison to approximately 70% in the comparison step in step S5 and in the determination step in step S8 and step S9 shown in FIG. 3. In other words, when the rate of reduction of fluorescence is monitored until the rate of reduction of fluorescence becomes 70%, it is possible to infer that the cells C are killed. In other words, it is possible to monitor the percentage of the cells C being killed.

In the case w here irradiation is performed with the therapeutic light L with the light intensity of 100 mW/cm² or more as shown by the solid line D in FIG. 4, reduction of fluorescence is also found whereas a rate of cellular cytotoxicity is low and hence, it is impossible to monitor cellular cytotoxicity by merely measuring the rate of reduction of fluorescence. In other words, the rate of reduction of fluorescence is not suitable as an index of the therapeutic effect.

However, in the case where irradiation is performed with the therapeutic light L with the light intensity of 50 mW/cm² or less as shown by the solid line B in FIG. 4, reduction of fluorescence and cellular cytotoxicity have a linear relationship similar to the linear relationship shown by the dashed-and-dotted line A. Therefore, it can be considered that when the rate of reduction of fluorescence is monitored, it is possible to monitor cellular cytotoxicity, that is, the rate of reduction of fluorescence can be used as the index of the therapeutic effect.

This is the basis for setting the light intensity with respect to the cells C to more than 0 mW/cm² and 50 mW/cm² or less in the light irradiation step in step S2 shown in FIG. 3.

Pan-IR700, which is the photosensitizing agent 20, is administered to the A431 tumor bearing mice at 300 μg/mouse. One day after the administration of Pan-IR700, the cells C are irradiated with light of respective irradiation intensities shown in FIG. 5 up to the same total amount (100 J/cm²). After one more day, tumor tissues are excised, and rates of damage of the tissues are calculated from pathological images of cross sections of the tumors. The chart of experimental data shown in FIG. 5 shows the results of the calculation of the rates of damage of the tissues.

As the result of the experiments performed by the applicant, it is found that, as shown in FIG. 5, when a comparison is made at the same irradiation energy density, cellular cytotoxicity when the light intensity is low is greater than cellular cytotoxicity when the light intensity is high.

More specifically, as the result of the experiments performed by the applicant, it is found that, in the case where the total amount is constant, cellular cytotoxicity when light irradiation is performed with the light intensity of 25 mW/cm² or 50 mW/cm² is greater than cellular cytotoxicity when light irradiation is performed with the light intensity of 100 mW/cm² or 300 mW/cm². Note that no data is acquired under the light intensity of 150 mW/cm².

Referring to details disclosed in Japanese Patent Application Laid-Open Publication No. 2017-71654, it is disclosed that a therapeutic effect depends on a total amount of light irradiation irrespective of a light intensity. However, as the result of the experiments performed by the applicant, it is found that a higher light intensity causes lower cellular cytotoxicity even when the total amount of light irradiation is constant.

This is the basis for setting the light intensity with respect to the cells C to more than 0 mW/cm² and 50 mW/cm² or less in the light irradiation step in step S2 shown in FIG. 3.

As described above, the present embodiment illustrates that when the cells C are irradiated with the therapeutic light L in PIT, the light intensity of the therapeutic light L is set to more than 0 mW/cm² and 50 mW/cm² or less, and the cells C are irradiated with light up to at least 1 J/cm².

With such a configuration, as shown in FIG. 4 and FIG. 5, when the cells C are irradiated with a predetermined energy in a state where the light intensity of the therapeutic light L is set to 50 mW/cm² or less, a great cell killing effect can be expected in PIT.

Further, even if the cells C are irradiated with the therapeutic light L with an intensity lower than a conventional intensity, it is possible to kill the cells C with certainty while an effect on a living body is reduced by a corresponding reduced amount of intensity.

Accordingly, it is possible to provide a method for irradiating cells with light, the method being capable of killing cancer cells with certainty by light irradiation in PIT.

In a conventional PIT, confirmation of whether the cells C are killed is made at a later date by making observation that uses an endoscope, for example. In a case where a therapeutic effect is not achieved, PIT is performed again.

To sufficiently exhibit the therapeutic effect on the cells C in PIT, it is necessary to irradiate the photosensitizing agent 20 with light with high intensity.

However, when the scene of irradiating the therapeutic light is observed with an endoscope, the tissue around the cancer cells in which the photosensitizing agent 20 is not accumulated reflects the therapeutic light, so that halation occurs in the endoscopic image.

Therefore, there is the following problem. In a state where the photosensitizing agent 20 is irradiated with the therapeutic light L, halation occurs and hence, not only that it is difficult to check a position where the photosensitizing agent 20 is irradiated with the therapeutic light L, but also that it is difficult to simultaneously monitor light irradiation and a rate of reduction of fluorescence. In addition to the above, it is also difficult to detect the rate of reduction of fluorescence.

Such a problem does not occur in the observation of the rate of reduction of fluorescence after light irradiation. However, it is known from the applicant's experiments that, under conditions of high light intensity as shown in FIG. 4, a correlation between a percentage of reduction of fluorescence and cellular cytotoxicity, that is, a therapeutic effect, is low. It is also known that it is difficult to monitor the therapeutic effect by monitoring the percentage of reduction of fluorescence at present.

Further, the above-mentioned Japanese Patent Application Laid-Open Publication No 2017-71654 discloses that the therapeutic effect depends on a total amount of light irradiation. However, as the result of the experiments, it is known that merely increasing the light intensity of light irradiation does not improve the therapeutic effect, that is, does not increase cellular cytotoxicity.

Therefore, a conventional technique requires to observe the therapeutic effect by using an endoscope or the like after PIT is performed. However, with the configuration of the present embodiment, the cells C can be irradiated with the therapeutic light L with an intensity lower than a conventional intensity and hence, it is possible to expect fluorescence intensity to be measured by using the objective optical system 4, the image pickup device 5, and the image processing unit 53 with a reduced effect of halation caused by irradiation with the therapeutic light L. Further, it is possible to measure fluorescence intensity in real time.

Therefore, it is possible to simultaneously perform treatment of the cells C by irradiation with the therapeutic light L and measurement of fluorescence intensity. Further, it is possible to easily visually recognize a part where the photosensitizing agent 20 accumulates in the cells C, so that such a part can be irradiated with the therapeutic light L with certainty whereby light therapy can be performed on the cells C with certainty.

Further, when the light intensity of the therapeutic light L is set to 50 mW/cm² or less as shown in FIG. 4, as described above, it is possible to monitor cellular cytotoxicity by monitoring the rate of reduction of fluorescence.

As the result of the monitoring, when the rate of reduction of fluorescence does not reach the predetermined value after irradiation is performed by the constant total amount, the cells C can be immediately irradiated with the therapeutic light L again during the treatment.

Accordingly, in addition to obtaining the above-mentioned advantageous effects of the present embodiment, it is possible to provide a method for irradiating cells with light, the method being capable of killing cancer cells by reliable light irradiation in PIT, allowing monitoring of a therapeutic effect by monitoring the rate of reduction of fluorescence, and also being capable of immediately perform PIT again.

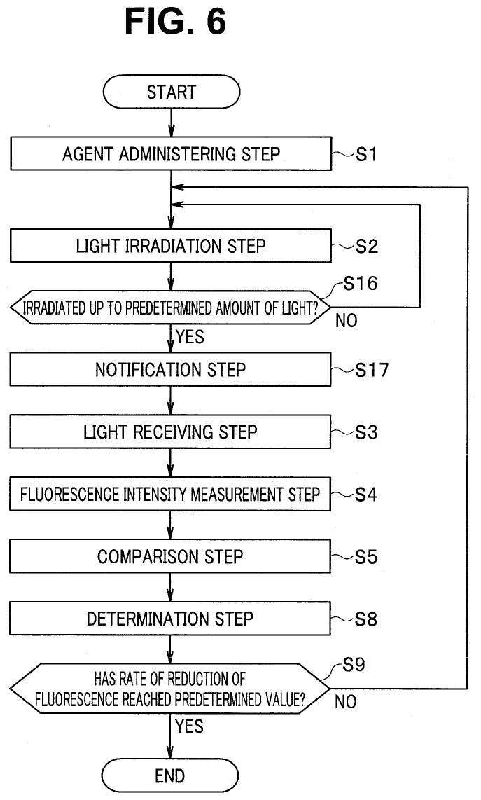

Hereinafter, a modification is described with reference to FIG. 6. FIG. 6 is a flowchart showing the modification where the fluorescence intensity measurement step in FIG. 3 is performed after the cells are irradiated with therapeutic light up to a predetermined amount of light.

The above-mentioned present embodiment illustrates that, in the fluorescence intensity measurement step, intensity data of fluorescence are acquired during the treatment where the cells C are irradiated with the therapeutic light L.

However, the acquisition of intensity data of fluorescence is not limited to the above. In the fluorescence intensity measurement step, intensity data of fluorescence may be acquired after the cells C are irradiated with the therapeutic light L up to the predetermined amount of light, that is, after the cells C are treated.

More specifically, as shown in FIG. 6, in performing PIT, first, the agent administering step is performed in step S1. In the agent administering step, the photosensitizing agent 20 shown in FIG. 2 is administered to the cells C.

Next, the light irradiation step is performed in step S2. In the light irradiation step, in a state where the illumination light I is supplied from the illumination optical system 2 to the cells C in the subject and within the observation range H of the objective optical system 4, the cells C are irradiated from the therapeutic light source unit 52 with the therapeutic light L with a light intensity (irradiation power density) of more than 0 mW/cm² and 50 mW/cm² or less by using the therapeutic light irradiation device 7 up to at least 1 J/cm².

Thereafter, in step S16, the image processing unit 53 determines whether the cells C are irradiated with the therapeutic light L up to the predetermined amount of light, more specifically, at least 1 J/cm².

When the cells C are not irradiated up to at least 1 J/cm², step S2 and step S16 are repeated.

In contrast, when the cells C are irradiated with the therapeutic light L up to at least 1 J/cm², the treatment is finished, and the process shifts to step S17 where the notification step is performed. In the notification step, the operator is notified that the cells C are irradiated with the therapeutic light L up to 1 J/cm². Examples of a specific notification method may be a known sound, light, display and the like.

Thereafter, the process shifts to step S3 where the light receiving step is performed. In the light receiving step, the image pickup device 5 receives, from the cells C, fluorescence that is generated from the photosensitizing agent 20 with irradiation of the therapeutic light L.

Next, the fluorescence intensity measurement step is performed in step S4. In the fluorescence intensity measurement step, the image processing unit 53 measures intensity data of the fluorescence.

Thereafter, the comparison step is performed in step S5. In the comparison step, the image processing unit 53 compares the intensity data of the fluorescence with the predetermined value. More specifically, the comparison step is performed where the image processing unit 53 makes a comparison, based on the intensity data of the fluorescence, to determine whether a percentage of attenuation of fluorescence (a rate of reduction of fluorescence) exceeds approximately 70% being the predetermined value.

Next, the determination step is performed in step S8. In the determination step, when the rate of reduction of fluorescence acquired from the intensity data is less than approximately 70% after irradiation with the therapeutic light L, the image processing unit 53 determines that the cells C should be irradiated with the therapeutic light L again until the rate of reduction of fluorescence becomes approximately 70%, and the image processing unit 53 gives an instruction to the therapeutic light source unit 52. In contrast, when the rate of reduction of fluorescence reaches approximately 70%, the image processing unit 53 determines that further irradiation with the therapeutic light L is unnecessary.

In following step S9, when the rate of reduction of fluorescence does not reach approximately 70%, it is determined that the therapeutic effect is low, and the process returns to step S2 and step S2, step S16, step S17, step S3, step S4, step S5, step S8, and step S9 are repeated. In other words, irradiation with therapeutic light is performed again.

In contrast, when the rale of reduction of fluorescence reaches approximately 70%, it is determined that the therapeutic effect is achieved with respect to the cells C, that is, the cells C are killed. Therefore, irradiation with the therapeutic light L is not performed again.

As described above, even when the intensity data of the fluorescence are acquired after the cells C are treated, it is possible to infer a percentage of the cells C being killed from the rate of reduction of fluorescence.

That is, it is possible to monitor the percentage of the cells C being killed by monitoring the rate of reduction of fluorescence and hence, it is possible to obtain advantageous effects substantially equal to the advantageous effects of the above-mentioned present embodiment. Other advantageous effects are equal to the corresponding advantageous effects of the above-mentioned present embodiment.

The description has been made with respect to a mode where the intensity of the therapeutic light L is set to a predetermined light intensity simultaneously with the start of irradiation. However, the mode is not limited to such a mode. For example, a light irradiation method may be adopted where a light intensity is gradually increased in such a manner that the light intensity reaches the predetermined light intensity after one minute from the start of irradiation. Alternatively, a light irradiation method may be adopted where a light intensity is gradually reduced from one minute before the finish of irradiation when light irradiation is finished. Gradually increasing or reducing light is expected to cause less damage to normal cells.

The image processing unit 53, which is the control unit, includes a processor including a central processing unit (CPU), storage devices such as a ROM and a RAM. All or a part of the configurations of a plurality of circuits of the processor may be executed by software. For example, the CPU may read and execute various programs that are stored in the ROM and that correspond to respective functions.

Further, all or a part of the functions of the processor may be achieved by a logic circuit or an analog circuit. Processing of various programs may be implemented by an electronic circuit, such as FPGA.

What is claimed is:

1. A method for irradiating cells with light, the method comprising:
administering a photosensitizing agent that uses a phthalocyanine dye, which is a fluorescent dye, to cells; and
irradiating the cells with predetermined light with a light intensity of more than 0 mW/cm² and 50 mW/cm² or less up to at least 1 J/cm².

2. The method for irradiating cells with light according to claim 1, wherein the photosensitizing agent includes IRDye 700 which is a phthalocyanine fluorescent dye.

3. The method for irradiating cells with light according to claim 1, the method further comprising:
notifying that the cells are irradiated with the predetermined light up to at least 1 J/cm².

4. The method for irradiating cells with light according to claim 1, the method comprising:
after irradiation of the cells with the predetermined light, receiving fluorescence that is generated from the cells with the irradiation with the predetermined light;
measuring intensity data of the fluorescence after the irradiation with the predetermined light is started; and
comparing the intensity data of the fluorescence with a predetermined value.

5. The method for irradiating cells with light according to claim 4, the method comprising:
after irradiation with the predetermined light up to at least 1 J/cm², determining to continue to irradiate the cells with the predetermined light up to the predetermined value in a case where the rate of reduction of fluorescence acquired from the intensity data is less than the predetermined value, and determining that further irradiation with the predetermined light is unnecessary in a case where the rate of reduction of fluorescence reaches the predetermined value.

6. The method for irradiating cells with light according to claim 1, the method comprising:
receiving fluorescence that is generated from the cells with irradiation with the predetermined light during the irradiation with the predetermined light;
measuring intensity data of the fluorescence generated with the irradiation with the predetermined light; and
comparing the intensity data of the fluorescence with a predetermined value.

7. The method for irradiating cells with light according to claim 6, the method comprising:
after the irradiation with the predetermined light up to at least 1 J/cm², determining to continue to irradiate the cells with the predetermined light up to the predetermined value in a case where a rate of reduction of fluorescence acquired from the intensity data is less than the predetermined value, and determining that further irradiation with the predetermined light is unnecessary in a case where the rate of reduction of fluorescence reaches the predetermined value.

8. The method for irradiating cells with light according to claim 4, wherein
in measuring the intensity data of the fluorescence, the intensity data are acquired after treatment by the irradiation with the predetermined light is finished.

9. The method for irradiating cells with light according to claim 1, wherein
the rate of reduction of fluorescence is a parameter having a correlation with cellular cytotoxicity in a case where the photosensitizing agent that uses the phihalocyanine fluorescent dye is used and the light intensity of the predetermined light falls within a range of more than 0 mW/cm² and 50 mW/cm² or less, the cellular cytotoxicity being a percentage of the cells that are killed or damaged with irradiation with the predetermined light.

10. The method for irradiating cells with light according to claim 1, wherein
the photosensitizing agent that uses the phthalocyanine dye is a fluorescent agent where a lower light intensity of the predetermined light causes greater cellular cytotoxicity, the cellular cytotoxicity being a percentage of the cells that are killed or damaged with irradiation with the predetermined light.

11. A method for controlling a medical dev ice capable of irradiating cells with therapeutic light, the method comprising:
irradiating cells to which a photosensitizing agent is administered with predetermined light with a light intensity of more than 0 mW/cm² and 50 mW/cm² or less up to at least 1 J/cm², the photosensitizing agent using a phthalocyanine dye, which is a fluorescent dye.

12. The method for controlling a medical device according to claim 11, the method comprising:
receiving fluorescence that is generated, after irradiation of the cells with the predetermined light, from the cells with the irradiation with the predetermined light;
measuring intensity data of the fluorescence after the irradiation with the predetermined light is started; and
comparing the intensity data of the fluorescence with a predetermined value.

13. The method for controlling a medical device according to claim 12, the method comprising:
after irradiation with the predetermined light up to at least 1 J/cm², determining to continue to irradiate the cells with the predetermined light up to the predetermined value in a case w here a rate of reduction of fluorescence acquired from the intensity data is less than the predetermined value, and determining that further irradiation with the predetermined light is unnecessary in a case where the rate of reduction of fluorescence reaches the predetermined value.

14. The method for controlling a medical device according to claim 11, the method comprising:
receiving fluorescence that is generated from the cells with the irradiation with the predetermined light during the irradiation with the predetermined light,
measuring intensity data of the fluorescence during the irradiation with the predetermined light; and
comparing the intensity data of the fluorescence with a predetermined value.

15. The method for controlling a medical device according to claim 14, the method comprising:
after irradiation with the predetermined light up to at least 1 J/cm², determining to continue to irradiate the cells with the predetermined light up to the predetermined value in a case where a rate of reduction of fluorescence acquired from the intensity data is less than the predetermined value, and determining that further irradiation with the predetermined light is unnecessary in a case where the rate of reduction of fluorescence reaches the predetermined value.

16. A medical device capable of irradiating cells with therapeutic light, the medical device comprising:
a therapeutic light source configured to irradiate cells to which a photosensitizing agent is administered with predetermined light with a light intensity of more than 0 mW/cm² and 50 mW/cm² or less, the photosensitizing agent using a phthalocyanine dye, which is a fluorescent dye; and
a therapeutic light irradiation device configured to control the therapeutic light source to perform irradiation with the predetermined light up to at least 1 J/cm².

17. The medical device according to claim 16, further comprising:
an image pickup apparatus configured to receive fluorescence that is generated, after irradiation of the cells with the predetermined light, from the cells with the irradiation with the predetermined light; and
a processor configured to measure, after the irradiation with the predetermined light is started, intensity data of the fluorescence, and compare the intensity data of the fluorescence with a predetermined value.

18. The medical device according to claim 17, wherein
after irradiation with the predetermined light up to at least 1 J/cm², the processor determines to continue to irradiate the cells with the predetermined light up to the predetermined value in a case where a rate of reduction of fluorescence acquired from the intensity data is less than the predetermined value, and the processor determines that further irradiation with the predetermined light is unnecessary in a case where the rate of reduction of fluorescence reaches the predetermined value.
